(19) United States
(12) Patent Application Publication
(10) Pub. No.: US 2017/0051335 A1
(43) Pub. Date: Feb. 23, 2017

(54) APPARATUS AND METHOD FOR THERMOCYCLIC BIOCHEMICAL OPERATIONS

(71) Applicant: J. Bruce HOOFNAGLE, Lisbon, MD (US)

(72) Inventors: Nelson NAZARETH, Upper Dean (GB); David EDGE, Warlingham (GB); Adam TYLER, Burton Latimer (GB)

(21) Appl. No.: 15/330,041

(22) PCT Filed: Jan. 28, 2015
(86) PCT No.: PCT/GB2015/000029

§ 371 Date: Jul. 29, 2016

(30) Foreign Application Priority Data
Jan. 29, 2014 (GB) 1401584.6

(51) Int. Cl. C12Q 1/68; B01L 7/00; G01N 21/64

(52) U.S. Cl. CPC C12Q 1/686; G01N 21/6452; G01N 21/6428; C12Q 1/6818; B01L 7/52; G01N 2021/6417; G01N 2021/6439; G01N 2201/068; B01L 2300/0654; B01L 2300/18; B01L 2300/0829

(57) ABSTRACT

Process and apparatus for the optimization of DNA detection and comprising: charging a plurality of reaction vessels with reagents and primers suspected of being suitable for the particular sample, in various quantities; placing in each reaction vessel a sample of the target DNA; subjecting each vessel concurrently to PCR; simultaneously observing optically the whole PCR process in each reaction vessel.

### FIELD OF THE INVENTION

The present invention relates to the identification of DNA. It is particularly concerned with the identification of pathogenic DNA in a context where time is of the essence on the one hand, and with the optimisation of a polymerase chain reaction (PCR) process for any particular target DNA on the other.

### BACKGROUND TO THE INVENTION

Generally speaking, PCR is performed on a DNA sample in order to check whether the sample contains a particular DNA whose presence is suspected, likewise RT-PCR for RNA species. Normally a sample is prepared for PCR by placing in a reaction vessel the necessary reagents and labelled primers. Then PCR is carried out by cyclically heating to a denaturing temperature, when the sample DNA strands separate, cooling to an annealing temperature where the separated strands bind with a primer, and heating to an extension point where the strands extend to make a new portion of the DNA. Thus at each cycle the target DNA, if present, doubles. Eventually the quantity is sufficient for detection, that is, assurance that the target DNA is indeed present. Optical reader means can observe the fluorescence generated when the DNA sample has been sufficiently amplified.

The PCR process has been incorporated into many molecular diagnostic tests but there remain still a vast, and in fact due to mutation, increasing number of molecules of interest. Thus there is a clear need both to rapidly establish both new tests, for example in the case of a disease outbreak situation, and to complete existing tests in the shortest possible time to detection, particularly when lives are at risk.

It is the case that every aspect of the PCR process is particular to the target DNA. Thus optimisation of the PCR process, including the rapidity with which it can be performed, may involve an extremely large number of iterations which, if performed consecutively might take many days, even weeks. The present invention aims to provide that these iterations can be performed largely concurrently in an automated operation, moreover one in which the results from each of the concurrent tests can be compared automatically to arrive at an optimum PCR process for a given combination of target DNA and primers. Not only could such an approach reduce the time taken to detection but ultimately examine the kinetics of the PCR process itself.

### SUMMARY OF THE INVENTION

According to a first aspect of the present invention a process for the optimisation of DNA detection comprises:

charging a plurality of reaction vessels with reagents and primers suspected of being suitable for a particular sample containing a target DNA, in various quantities;

placing in each reaction vessel the target DNA;

subjecting each vessel concurrently to differing thermal cycling profiles;

concurrently and continuously observing optically, usually via fluorescence, the whole PCR process in the reaction vessel.

From comparison of the results from each reaction vessel can be determined the optimum PCR process for a particular DNA target. Usually it may be necessary to excite with appropriate light the contents of the reaction vessel for corresponding fluorescence signal to be generated therein. By “continuously” is meant capturing images at intervals of less than one second, preferably 25 ms (milliseconds).

The quantity of reaction vessels is conveniently 96 in the customary 8×12 microtitre vessel array, and the timing of the process in each vessel is varied, possibly in accordance with the results obtained from the optical apparatus. With full control of both temperature and time it becomes possible for the instrument to run pre-programmed protocols. Thus the instrument can complete gradients in temperature versus time, a different gradient at that, perhaps, in each reaction vessel. Then, by comparing cT (cycle threshold) values, and R (statistical value relating to scatter with respect to a straight line) there can be determined by comparisons of these data the optimum conditions for that particular DNA.

Further, it becomes possible to study the enzyme kinetics of the reaction with respect to any of the variables. For example if two reactions are identical aside from primer concentration then if fluorescence increase is continuously observed, as opposed to a traditional once per cycle approach, then It is possible to determine the Km of the enzyme with respect to primer concentration. It is therefore possible to study the impact of all of these variables in the reaction in essentially a stepwise fashion but on a single plate—fixing all variables bar a single to be tested.

A number of phenomena occur which directly impact the observed fluorescence. Chief amongst these when viewed following the addition of an intercalating dye are the annealing and melt points of the target sequence. A system capable of spectrographic interrogation can observe the emitted fluorescence from both an intercalating dye and a sequence specific probe at exactly the same time and temperature. This enables measuring the FRET (fluorescent resonance energy transfer) and hence can provide information about the hybridisation state of the target. Further, because all data can be collated on a millisecond timescale it is not necessary to hold the cycle at any temperature for more than a few milliseconds after observation of the change or signal.

Likewise with the same primers in each well it becomes possible to study different aspects of the process by having differing intercalators or specific probes in each well and as such gain data on different aspects of the assay in a single run—thus determining for example the melt point in one well and the annealing point in another.

The above aspects give rise to a novel parameter which the inventors call in-cycle efficiency, which is in essence the Km of the enzyme under the specified reaction conditions—the higher this value is the quicker a reaction can be performed. When, as has hitherto been the case, there is a single reading per cycle a real-time PCR curve is generated for the whole PCR process. However when, by means of this present invention, the whole of any single cycle is observed there is additional and valuable information. Instead of the plot being just from the baseline fluorescence observed in any cycle, which reflects the points at which the cycle has been observed to have been complete, there is also available a plot of the moments when doubling has been completed. Thus are provided two datum points: firstly the time point at which the doubling has completed and secondly the slope of the curve described by the whole sequence of cycles. The slope of this curve we have termed the in-cycle efficiency factor and it will be appreciated that the point at which the doubling has completed represents the minimum possible time required for the amplification step.

If an intercalating dye is employed it is possible to observe all of denaturation, annealing and extension temperatures against time, and with high resolution annealing at that. High resolution annealing represents a novel approach to discriminating between similar sequences and being additionally able to quantify their relative abundance. If it is possible to visualise the point at which primers anneal, this is the point where the in cycle amplification signal has an initial spike when a suitable probe is employed, then it is possible to discriminate between two amplicons with similar annealing temperatures. Again, key here is the ability afforded by the invention to measure fluorescence within a time scale of just a few milliseconds—thus providing very high resolution, in theory 0.04° C. when performing a reading every 25 ms and cooling at 1° C./s. This is novel data that cannot be gathered by the traditional once per cycle reading at extension point, yet it adds no additional time requirement to the PCR protocol and removes the need for downstream confirmatory methods such as high resolution melting.

The invention makes possible a rapid factorial optimisation of the process for identifying a particular DNA. Among the parameters susceptible of optimisation are:

anneal temperature;

annealing time

denaturation temperature;

denaturation time;

extension temperature;

extension time;

temperature at which fluorescence readings are taken;

ramping rates (for all steps);

magnesium chloride concentration;

dNTP concentration;

primer concentration;

target concentration;

enzyme concentration.

Of these, the most important may be anneal temperature; extension time; magnesium chloride concentration; and primer concentration.

The optimisation of any one of these parameters is dependent upon the effects of the other, and yet other, parameters. If the extension time is too short the process efficiency, including the cT and R values, will drop, meaning that the DNA sample will not double in each cycle.

If the selected annealing temperature is too high then not all priming sites will be covered and once again process efficiency falls.

Again if the concentration of either magnesium chloride or the primer is too low then the replication complex deteriorates and as such the in-cycle efficiency will have dropped.

Factorial optimisation according to the present invention operates to test the impact of making individual changes to the above parameters and determine which parameter combination will result in the lowest cT and a value of R closest to 1. The additional in cycle efficiency factor, in essence the Km of this enzymatic process, is also utilised in order to maximise efficiency and minimise time to detection.

In outline the process for factorial optimisation is as follows:

The user is supplied with a 96 vessel plate either as a consumable or with instruction as to which reagents are to be placed at which concentration in each position. In the preferred embodiment the plate is supplied as a consumable item such that the reaction contents are highly reproducible and tightly controlled. The user simply adds the primers, probes and targets at prescribed concentration as instructed and the plate is sealed ready for thermal cycling. The instrument, having full independent well control and monitoring, operates a pre-programmed thermal cycling profile across the reaction vessels. As to the spectroscopic aspect of this embodiment, the temperature and fluorescence readings are tied intimately together. This is because many of the assays have a multiplexed component and hence need to acquire two dyes concurrently and continually for the iPCR process. It will be clear that a standard filter based approach and with a set of filters to discriminate dyes can never meet these performance requirements. The instrument will then record the full fluorescence spectrum obtained for each vessel with a frequency of under 1 second. Once completed the instrument has software programmed to take the raw spectral data, spectrally deconvolute, to separate the fluorescence attributed to each individual component dye. The software is then able to plot the required graphs, including fluorescence against time, against temperature and also efficiency against each individual reagent concentration. An example is; if the profile has 4 identical reaction vessels, the same thermal profile, the same reagents other than for example primer concentration, a plot of the relative in cycle efficiencies would give a bell curve and the software can determine the optimal concentration by interrogating these data. The system can then supply the user a full list of the ideal time/temp/concentration of each assay and further can suggest an ideal optimised PCR. The process is termed factorial optimisation and is a key benefit of the intelligent PCR approach, namely rapid independent well control of the thermal system and high frequency “continuous” spectrographic interrogation of the reactions.

By extension the system should be capable of taking any existing assay and performing this form of optimisation with regards to only the temperature and time aspects. For example total reaction time may be minimised by automatically moving onto the next cycle when fluorescence doubling is observed. Further, the system could additionally perform such optimisation with a single well by running different profiles each cycle in order to reduce reaction time.

In summary, the intelligent PCR approach is to leverage the technical advantages arising from the use of independently controlled and monitored thermal cycling when combined with the ability to spectrographically interrogate those same wells on a sub second timescale. This generates novel data that cannot be obtained by existing instrumentation and the intelligent PCR is the processes and methods arising from the use of this data.

According to a second aspect of the present invention there is provided apparatus for cyclic biochemical operations, including PCR, the apparatus comprising an array of microtitre reaction vessels, each individually controllable, a laser or laser diode light source, a multi-channel imaging spectrograph, a multi-fibre probe bundle arranged for the reception of a collimated output of the light source and terminating above at least eight reaction vessels, each fibre probe actually comprising a plurality of excitation fibres and at least one collector fibre, the said at least one collection fibre being arranged to be focussed, perhaps via diffraction grating, onto a large area detector.

Ideally the number of fibre bundles is 96 and the spectrograph is a 96 channel imaging spectrograph. In this way full spectral data can be continuously collected concurrently throughout all reactions. Where only eight fibre bundles are employed in the 96 well context there may be a moving shuttle arranged to centre the spectrograph over each column of 12 wells in turn. Or twelve bundles may be employed, with a shuttle arranged to centre the spectrograph over each row of eight wells in turn.

Preferably the light source is a laser or laser diode operating at 488 nm due to the use of green dyes being commonly used in molecular diagnostics. A cheaper light source utilises LEDs at a similar wavelength has also been tested. A multiplexer may also be employed. Preferably the entire bundle of 96 fibres is concurrently illuminated from a single 488 nm source.

According to a feature of this aspect of the invention each fibre probe end may contain a single central core arranged to collect the emitted light arising from the amplification taking place. Suitably in fact there may be a single central core collector fibre surrounded by six, this being geometrically perfect for fibres of the same diameter, excitation fibres. The emitted light is thus transmitted back to a similar multifibre bundle on a second leg of the photometer but in this case organised into a prescribed array such that this array can be focussed via a diffraction grating onto a large area detector such as a CCD. As a result a plurality of individual spectra are concurrently imaged on the CCD device and as such all emission light at any visible wavelength is collected from all 96 wells simultaneously or sequentially in multiples of eight or twelve.

In summary, a single laser (or laser diode) source can be arranged to provide a spectrally collimated high power source, optic fibre collection and delivery and concurrent high-speed imaging of all 96 vessels. In the preferred embodiment this is a 488 nm laser diode operating at 50 mw but other wavelengths and input powers could be utilised dependent on the dyes being used. The use of such a system makes possible the reading of a complete fluorescence spectra in 25 milliseconds but any full spectrum readings in a sub 500 ms time frame makes possible this approach.

The optical means can be arranged to capture the full visible spectrum from the wells, preferably at least eight at a time. The optics may comprise a single detector and rotary distribution wheel, an eight well scanning head, a spectral photometer capable of reading one to eight reaction vessels, preferably without moving, or an imaging spectrograph which can view all the reaction vessels at the same time, as described above. This latter is the much preferred optical means.

An eight well scanning head may comprise a single detector and two diffraction gratings to focus eight spectra onto the one sensor. Both excitation and emission light may be provided by fibres which feed into an eight well LED board and a spectrograph respectively. By this means a picture of the spectrum can be built up by capturing the individual bands. The 96 wells may be addressed by means of

The system comprises a novel rapid imaging spectrograph for the continual Spectral interrogation of real-time PCR reactions. Moreover, independently controlled ultra-rapid thermal cycling in for example 96 (12×8 array) microtitre reaction vessels combined with this rapid imaging, makes possible both automated optimisation of any assay but also the reduction of the time to detection of a target DNA to the absolute minimum.

In order to reduce the time taken to capture the spectra the number of spectrographs can be increased all the way up to 12 when no moving parts would be required. An alternative embodiment comprises means for imaging the whole 96 wells onto a camera and having a set of filters that can concurrently be placed in front of the lens.

With the imaging spectrograph embodiment the light emitting from each well is turned into a spectrum and focused on a large area detector. Detectors can be CCD or preferably CMOS. Excitation can be provided by means of 488 nm laser but preferably there can be used an LED (or LEDs) centred around this wavelength with cut off filters to remove unwanted portions of its emission. This forms the preferred embodiment of the apparatus for performing the iPCR method, including the factorial optimisation approach described therein.

It will be appreciated that in a microtitre context the plan space above each well available for the optics is a maximum of 9×9 mm.

By the resolution available from this invention it is possible to see both the time point and also the temperature at which the annealing step occurred and also which of the fluorescently bound molecules successfully annealed. It is then possible to discriminate between multiple alleles, such as SNP screening at any given locus as well as the standard quantification of the data. Designing a pair of primers to cover the region of interest differing in both melt point and fluorescent label permits the accurate determination of the temperature at which annealing occurs. This will be subtly different between the two. With the system capable of spectral deconvolution it could then separate the dyes spectrally but combine their total fluorescent output if required and also compare if necessary. By these means it becomes possible to genotype SNP variants whereas it has not been possible to design real-time PCR probes without significant cross-talk between the amplicons.

### BRIEF DESCRIPTION OF THE DRAWINGS

Embodiments of the invention will now be described by way of example with reference to the accompanying drawings, of which:

FIGS. 1 to 4 illustrate a 96 microtitre reaction vessel array with individual PCR control.

FIG. 5 is a schematic drawing of an array of fibre optic bundles;

FIG. 6 is a sectional view of one fibre optic bundle;

FIGS. 7 and 8 are graphs illustrating the advantage of “continuous” reading; and

FIGS. 9 to 16 are plan views of examples of plate layouts for factorial optimisation;

### SPECIFIC DESCRIPTION

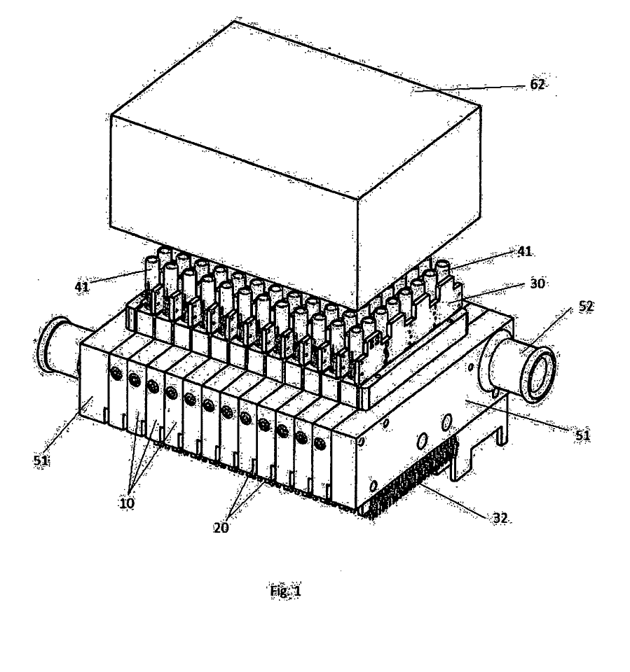

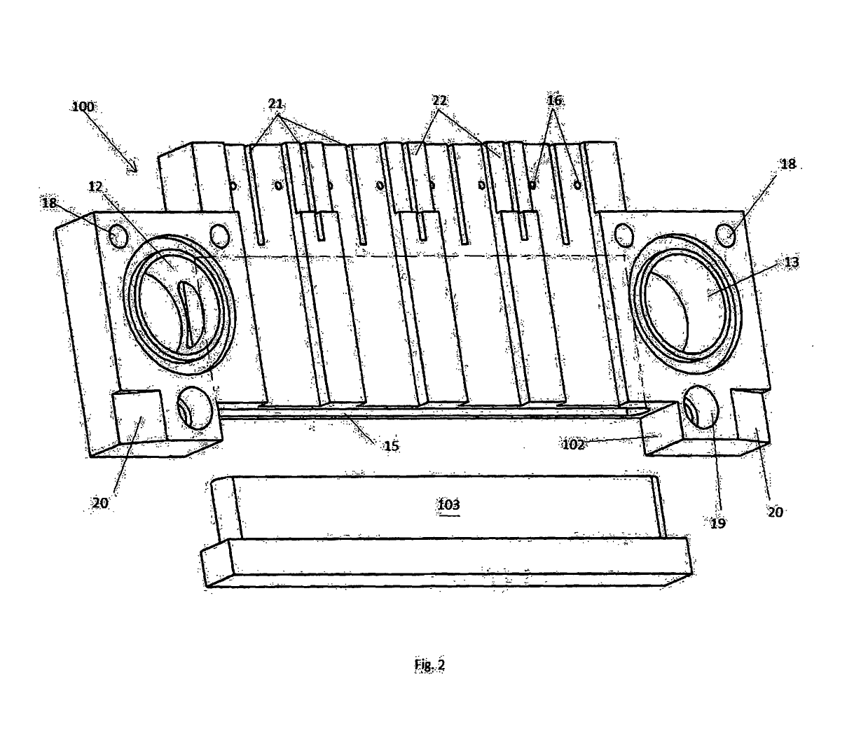

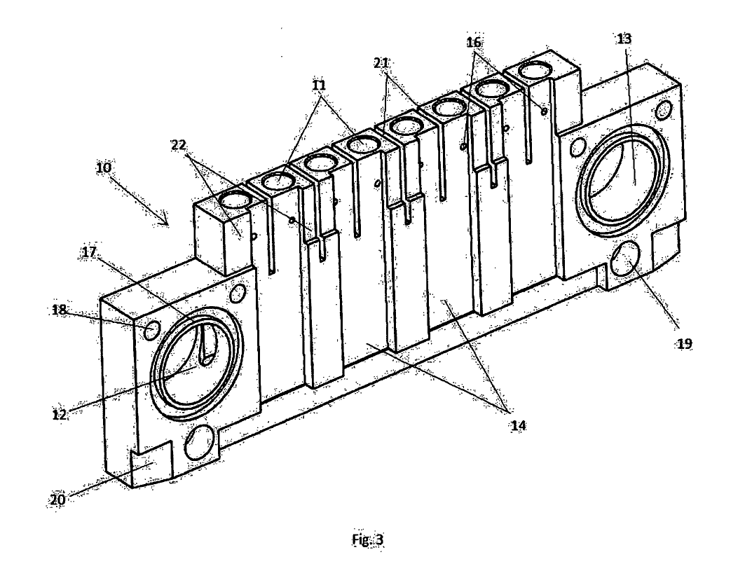

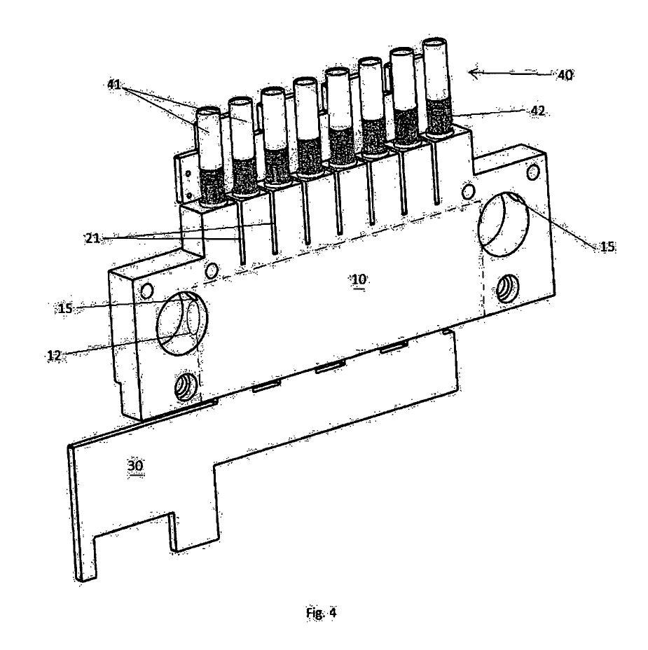

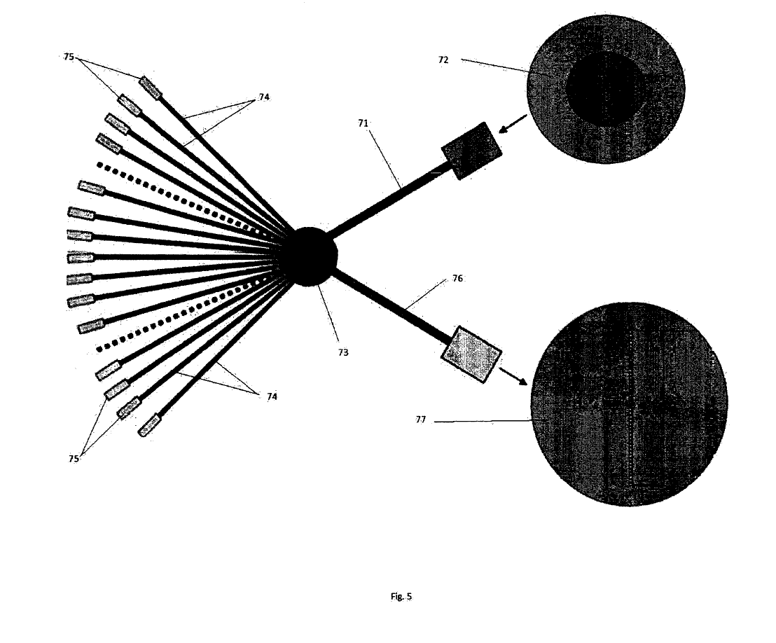

A 96 microtitre reaction vessel PCR apparatus in a standard 12×8 array is described in, among other patent specifications, those of UK Patent 2404883 and co-pending UK Patent Application 1401584.6, both of which describe individual well control. A resume of the latter is described below with reference to FIGS. 1 to 4.

The apparatus comprises twelve heat removal module slices 10 sandwiched between two end plates 51 having coolant liquid inlet and outlet necks 52, 53. Each slice has eight reaction stations 11 at a top edge, coolant liquid entry 12 and exit 13 manifold bores therethrough at each end, and a series of grooves 14 extending along one face from the top to the bottom edge thereof. A heat exchanger liquid hollow extends between the manifold bores 12 and 13.

The reaction stations 11 are circular hollows sized for the bases of reaction vessel holders 40 to be an interference fit therein. A small hole 16 leads from the base of each station 11 to the groove 14 and acts in use to permit the escape of gases (air) from the stations 11 when the vessel holders are driven in.

Around each manifold on one face of the slice are grooves 17 for an O-ring seal and further out are slide attachment holes 18 of which one has a locating hush 19.

At each bottom corner on one face is a separation rebate 20 arranged to assist in separating the slices when required. Between each station 11 there is a cut 21 arranged to maximise thermal isolation between each station 11. Rebates 22 on one side of each slice 10 are formed for a like purpose.

A printed circuit board (PCB) 30 clips into the grooves 14 and projects above and below the slice 10. The PCB 30 carries heater and sensor electrical conduits which terminate in connectors 31 at the top and 32 at the bottom thereof. The thickness of the PCB 30 is the depth of the grooves 14.

A reaction vessel holder 40 fits into each of the reaction stations 11. The reaction vessel holder 40 comprises a reaction vessel receiving portion 41; a heater portion 42 and a cooling portion 43, the latter being arranged to anchor the station in a heat removal module. The vessel receiving portion 41 is shaped to receive snugly a microtitre reaction vessel and in the wall thereof is located a temperature sensor 44. The heater portion 42 has a helical groove therearound into which is wound a heater coil 45. Flexible tubing (not shown) connects the necks 52, 53 with a heat sink coolant reservoir (not shown) via a pump (not shown).

The reaction vessel 61 is a microtitre vessel formed of a carbon loaded plastics material and is 2 cm overall length. It comprises, in descending order, a cap receiving rim, a filler portion and a reaction chamber with a base thereto. The filler portion has a maximum outer diameter of 7 mm and a depth of 5 mm. The reaction chamber tapers down from 3 mm to 2.5 mm, the whole having a wall thickness of 0.8 mm. Accordingly the reaction chamber is of substantially capillary dimensions.

The array of holders 40 is adapted to accept snugly a 12×8 standard microtitre well tray 60

During a reaction electrical supply via the conduits is arranged to heat the wells 61 according to a predetermined program, while other of the conduits convey signals relating to the temperature in the wells. This program is predetermined for each well, as the apparatus is particularly suited for performing totally independent reactions in each well 61. Thus, where the reactions comprises a heating-cooling cycle, as is the case for example in PCR, one well 61 may be in a heating phase and another in a cooling phase, one at rest and another complete.

The heating cycle is arranged to take place against a coolant environment in the HRM 50 which is fixed at 40° C. which is usually above room temperature and is a mid-point for heating and cooling efficiency.

The progress of the process in each reaction vessel is monitored in the optics unit 62

FIG. 5 illustrates an array of fibre optic bundles used in a 8×12 microtitre plate. A bundle of excitation fibres 71 emanate from a CCD light source 72 and pass into a multiplex unit 73 wherefrom emerge 96 fibre optic bundles 74 each comprising excitation fibres and at least one collection fibre. The bundles 74 each terminate in probes 75 destined to be mounted appropriately one above each reaction chamber. The collection fibres are connected in the multiplex unit 73 to an output bundle 76 which is passed to a spectrograph 77.

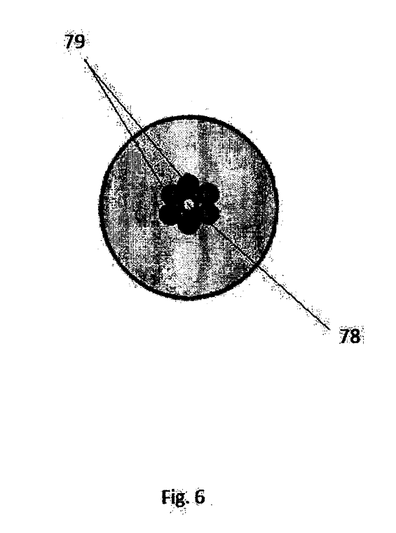

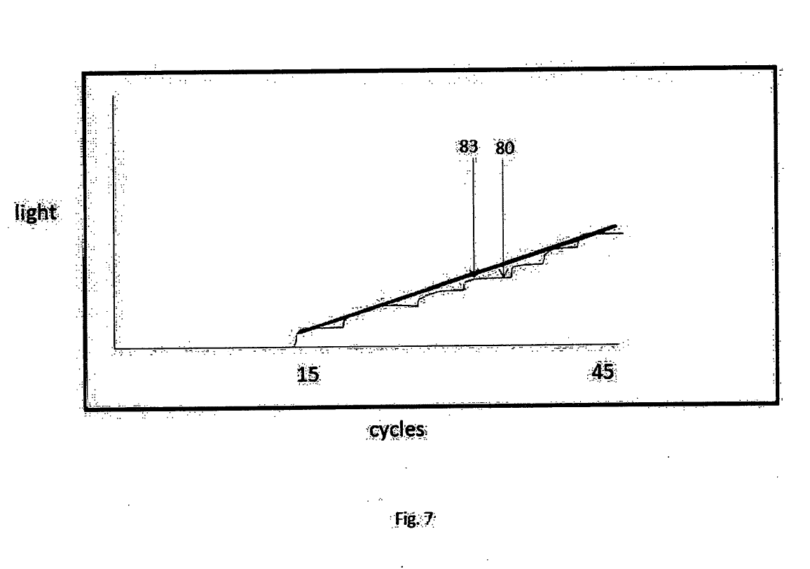

FIG. 6 is a sectional view of one fibre optic bundle 74, that is, a bundle emanating from the multiplex unit 73 and terminating in a probe 75. Each bundle 74 comprises a collection fibre core 78 and six excitation fibres 79 surrounding the core fibre 78. A standard protective shield surrounds the fibres.

It is the probes 75 which are in the optics unit 62 shown in FIG. 1, mounted with one probe 75 facing each well 61.

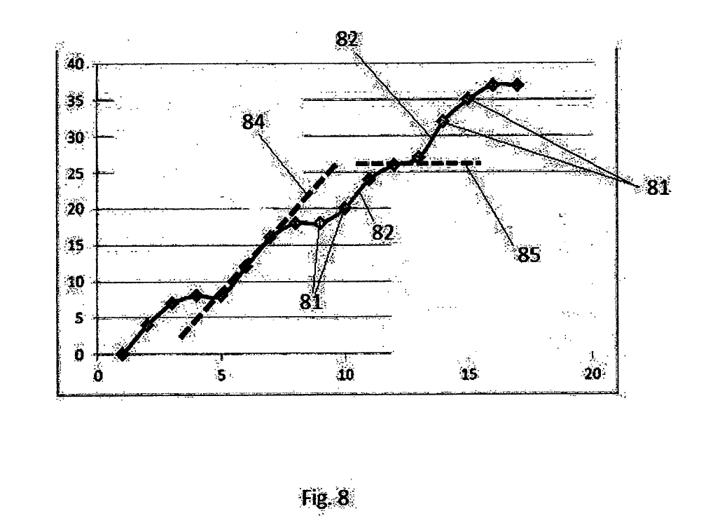

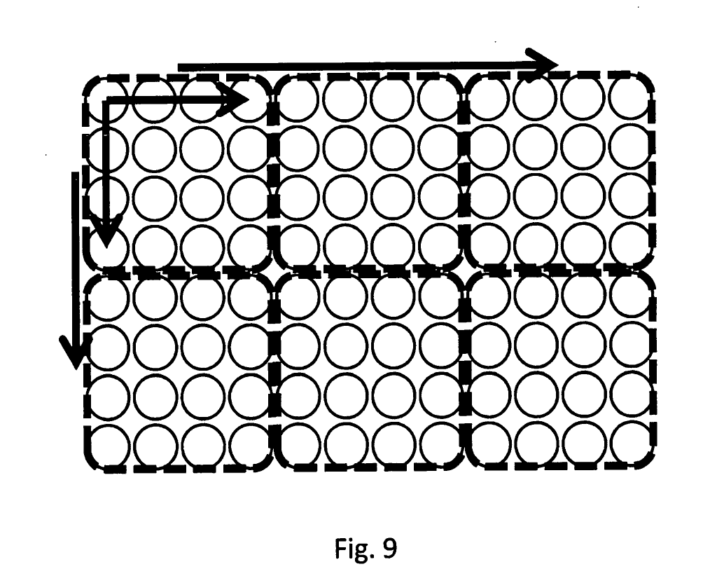

FIGS. 7 and 8 are graphs of light emission (y axis) versus the number of cycles (x axis). The graphs illustrate the difference between traditional PCR optical observation and that of the present invention with FIG. 8 illustrating a detail (four cycles) from FIG. 7. In the traditional optical observation wherein filters or movable probes are employed, a single image capture is made at the end of each cycle, that is, after each extension, of necessity. This is at point 80 in FIGS. 8 and 9. In continuous capture, that is, an image every 25 ms, images are captured at points 81, enabling the construction of a real time line 82 representing the whole PCR process. In particular the moment of extension can be captured (point 83) and slope angle and time length of each step, cT and R observed and optimised. The dashed line 84 provides accordingly a measure of in-cycle efficiency. The dashed line 85 is the measurement of the point at which doubling has completed.

Accordingly, when viewed in real time the data obtained makes possible the measurement of the point when amplification has been observed to have been completed for the given cycle. Any additional time on this cycle is unnecessary. Furthermore it is possible to visualise the in-cycle efficiency by measuring the slope (line 83)of the fluorescence increase within each cycle. Differing fluorescent chemistries, for example intercalating dyes and the 3′ hydrolysis assay, will give differing amounts of data on each of the segments of the reaction. The example shown is for a 3′ hydrolysis assay. An intercalator will also show the melt points of the DNA products and this will be of benefit to the automated software. By interrogating the same DNA target with different probe systems it is possible to build up a picture of the reaction in its entirety; annealing temperature, the effect of different chemical constituents, optimised temperatures, and hold times at the same.

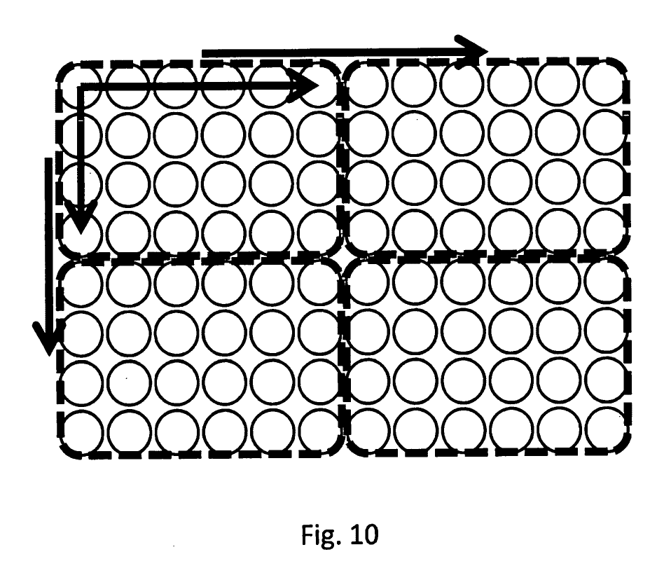

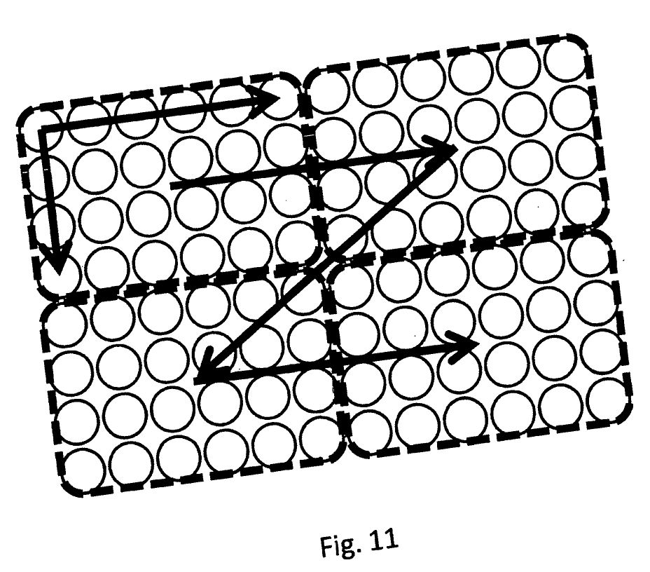

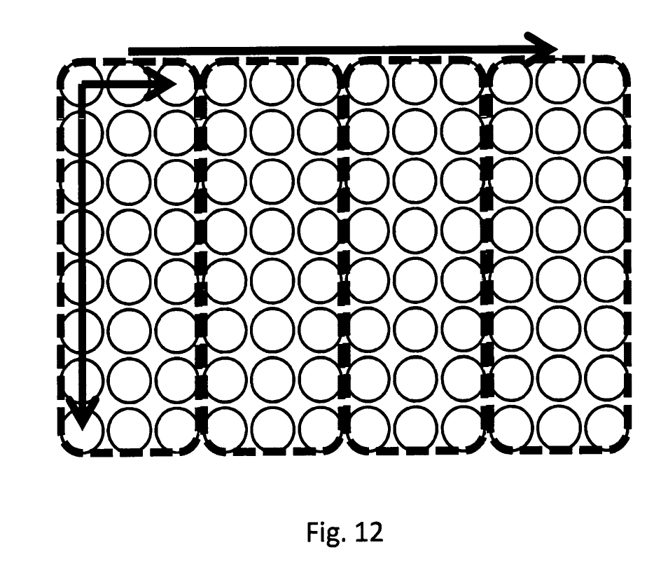

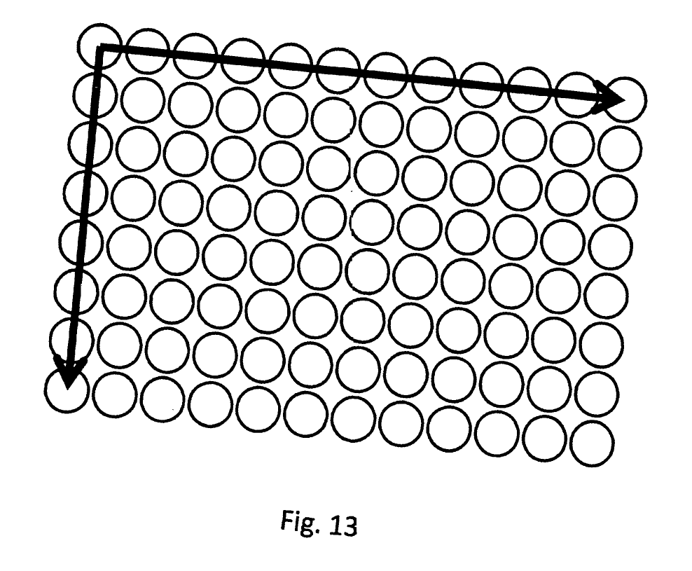

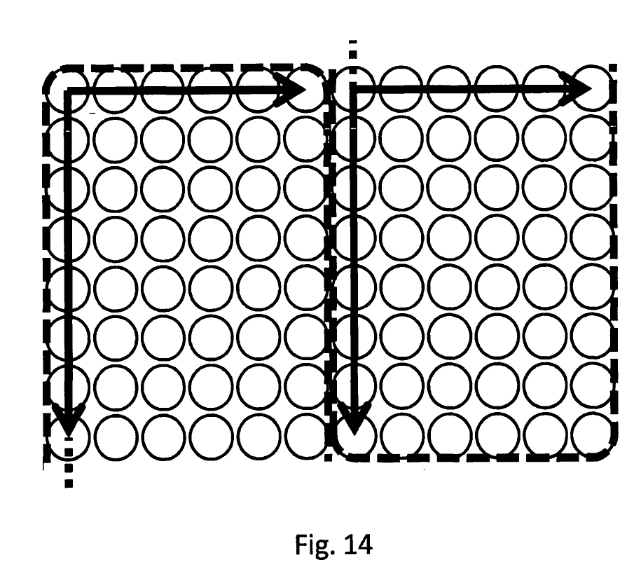

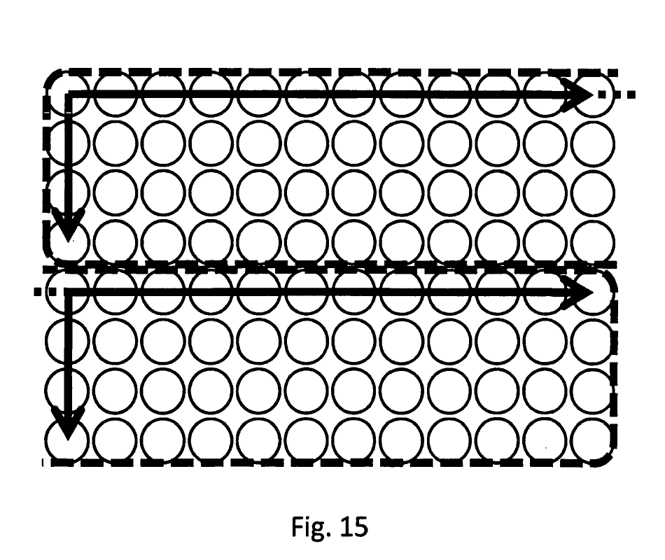

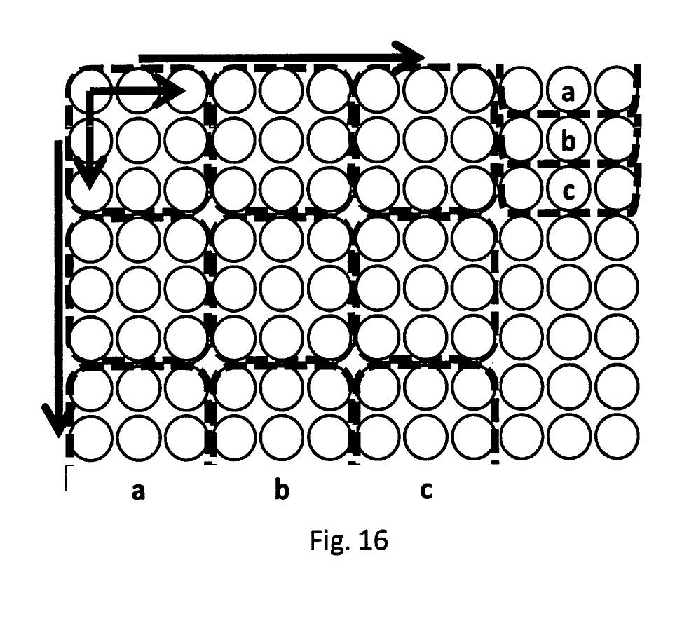

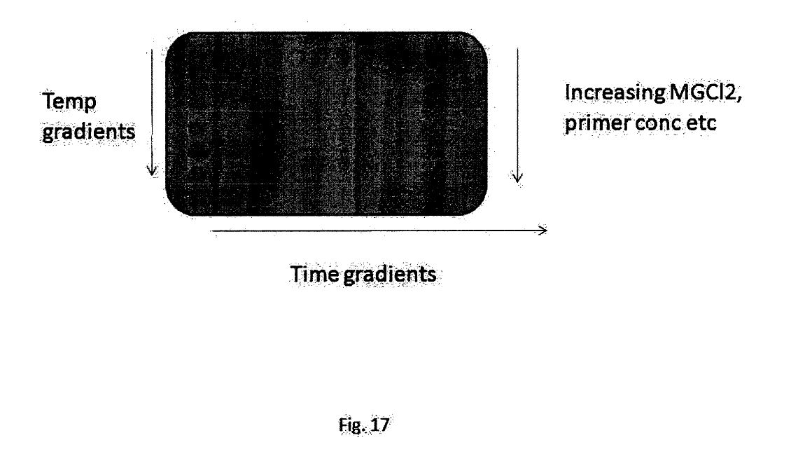

FIGS. 9 to 16 illustrate patterns of concurrent PCR operations in a standard 8×12 microtitre reaction vessel array, where the numbers cited represent one variable, e.g. annealing temperature; extension time; magnesium chloride concentration etc; Thus:
FIG. 9 shows an array set up for 4×4×3×2 concurrent tests;
FIG. 10 shows an array set up for 6×4×2×2 concurrent tests;
FIG. 11 shows an array set up for 6×4×4 concurrent tests;
FIG. 12 shows an array set up for 3×8×4 concurrent tests;
FIG. 13 shows an array set up for 12×8 concurrent tests;
FIG. 14 shows an array set up for 6×16 concurrent tests;
FIG. 15 shows an array set up for 24×4 concurrent tests; and
FIG. 16 shows an array set up for 3×3'3×3 concurrent tests.

By “set up” is meant that the array, in the art usually called a plate, is pre-prepared with the range of, for example, magnesium chloride, primer, enzyme and dNTP concentrations.

Then, in the course of the concurrent tests, time gradient can for example be varied on a column by column basis and temperature gradients can be varied on a row by row basis, as illustrated in FIG. 17.

1.-36. (canceled)

37. A process for the optimization of DNA detection comprising:
charging a plurality of reaction vessels with reagents and primers suspected of being suitable for the particular sample, in various quantities;
placing in each reaction vessel a sample of the target DNA;
subjecting each vessel concurrently to PCR;
concurrently observing optically the whole PCR process in each reaction vessel.

38. A process as claimed in claim 37 and arranged to examine at least several of the following parameters:
anneal temperature;
annealing time
denaturation temperature;
denaturation time;
extension temperature;
extension time;
temperature at which fluorescence readings are taken;
ramping rates (for all steps);
magnesium chloride concentration;
dNTP concentration;
primer concentration;
target concentration.

39. A process as claimed in claim 37 and comprising spectrographic interrogation of the emitted fluorescence from both an intercalating dye and a sequence specific probe at the same time and temperature, thus measuring the FRET and hence providing information about the hybridisation state of the target.

40. A process as claimed in claim 37 and comprising spectral deconvolution to separate the individual component dyes and comparing their total fluorescent output.

41. A process as claimed in claim 37 and arranged to discriminate between highly similar sequences and comprising designing a pair of primers to cover the region of interest, and placing these primers in the same reaction vessel, the primers differing in both melt point and fluorescent label and thus determining the actual temperature at which annealing occurs and enabling the required discrimination.

42. A process as claimed in claim 37 and wherein optical means are arranged to capture the full visible spectrum from each of the reaction vessels.

43. A process as claimed in claim 42 and further comprising separating the fluorescence arising from each of the reaction vessels, plotting the fluorescence values against time, temperature and concentration of each assay and indicating an ideal optimised PCR.

44. A process as claimed in claim 37 and wherein the reaction vessels are in an 8×12 microtitre vessel array.

45. A process as claimed in claim 37 and comprising determining automatically, from the results in each reaction vessel, the most rapid and efficient identification process for a given DNA target, and indicating same.

46. Apparatus for carrying out the process of claim 37, the apparatus comprising an array of icrotiter reaction vessels, means for performing polymerase chain reaction each in each reaction vessel concurrently on an individual basis, a light source, a multi-channel imaging spectrograph, means for controlling the time of the PCR, a multi-fiber probe bundle arranged for excitation and the reception of a collimated output of the light source and terminating above at least eight reaction vessels, each fiber probe actually comprising a plurality of excitation fibers and at least one collector fiber, the said at least one collection fibre being arranged to be focused onto a large area detector.

47. Apparatus as claimed in claim 46 and wherein the at least one collection fiber is focused onto the detector via a diffraction grating,

48. Apparatus as claimed in claim 46 and wherein the means for performing polymerase chain reaction on the contents of the reaction vessels comprises a heater, a heat removal module, a heat sink coolant reservoir and a pump.

49. Apparatus as claimed in claim 46 and wherein the light source is a laser or laser diode.

50. Apparatus as claimed in claim 46 and employing an optical multiplexer.

51. Apparatus as claimed in claims 46 and wherein the spectrograph is arranged to capture the full visible spectrum from the wells.

52. Apparatus as claimed in claim 46 and comprising an array of 96×n, where n is an integer, microtitre reaction vessels in 12×8 array, at least a plurality of which are arranged for individual control and further comprising an eight well scanning head having a single detector and two diffraction gratings to focus eight spectra onto the one sensor.

53. Apparatus as claimed in claim 46 and wherein the optical means comprises a single detector and rotary distribution wheel, an eight well scanning head, a spectral photometer capable of reading one to eight reaction vessels, preferably without moving, or an imaging spectrograph which can view all the reaction vessels at the same time, as described above.

54. Apparatus as claimed in claim 46 and further comprising a shuttle arranged to center the spectrograph over each column of wells in turn.

55. Apparatus as claimed in claim 46 and wherein each fibre bundle comprises a single central core collector fiber surrounded by six excitation fibres.

56. Apparatus as claimed in claim 46 and wherein the large area detector is a CCD or a CMOS.

57. Apparatus as claimed in claim 53 and wherein the eight well scanning head comprises a single detector and two diffraction gratings to focus eight spectra onto the one sensor.
